(19) United States
(12) Patent Application Publication
(10) Pub. No.: US 2024/0382868 A1
(43) Pub. Date: Nov. 21, 2024

(54) EXTRACTION

(71) Applicant: 113 Botanicals Limited, Southend On Sea (GB)

(72) Inventor: Mazin NICOLA, Sussex (GB)

(21) Appl. No.: 18/688,834

(22) PCT Filed: Aug. 12, 2022
(86) PCT No.: PCT/GB2022/052115

§ 371 Date: Mar. 4, 2024

(30) Foreign Application Priority Data
Sep. 2, 2021 (GB) 2112506.7

(51) Int. Cl. B01D 11/02

(52) U.S. Cl. CPC B01D 11/0288; B01D 11/0207; B01D 11/0296; B01D 2311/103; B01D 2311/106

(57) ABSTRACT

Extraction Apparatus (2) for extraction of a biomass comprises an extraction vessel (4) for containing a biomass. Electrical heating tape (6) is provided around the vessel (4) for maintaining the temperature of the wall of vessel (4). Upstream of vessel (4) is a solvent recycling vessel (8) which is fitted with a cooling coil (10) which communicates with an external refrigeration unit (12). Operation of unit (12) is arranged to cool (and thereby liquefy) solvent in the vessel (8). A solvent pump (22) is arranged to pump liquid between vessel (8) and vessel (4) via an electrically powered heat exchanger (24). The heat exchanger (24) is arranged to increase the temperature of solvent flowing through it very rapidly which allows the temperature of solvent introduced into extraction vessel (4) to be very rapidly changed in a step-wise manner.

This invention relates to extraction and provides apparatus for use in extraction, a system comprising such apparatus and method of extraction. Preferred embodiments relate to extraction of active materials from biomasses, for example from cannabis plant material, for example from cannabis sativa.

Cannabis Sativa is believed to be one of the oldest plants cultivated by man. The plant and preparations therefrom have been used for its medicinal properties for many centuries. However, the real breakthrough in understanding the potential of medicinal properties of cannabis occurred in the 1990s following the discovery of the endocannabinoid system (ECS) in the human brain and other parts of the body. The ECS is composed of endocannabinoids which are lipid-based neurotransmitters, or G proteins, that bind to cannabinoid receptors, CB1 and CB2. Endocannabinoids are thought to play a role in regulating a variety of physiological processes including, for example mood control, memory, pain sensation and energy metabolism. It is now universally accepted that the bioactivity of cannabinoids is attributed to their interactions with the endocannabinoid system in the physiology of the human body.

The easing of some of the prohibition laws relating to the cultivation and use of cannabis material started with “California Proposition 215” also known as The Compassionate Use Act in 1996 and by 2016 medical use of cannabis was legalised in most of the USA. This resulted in the ever-increasing global demand for cannabis plant products for recreational use and as health supplement and homeopathic medicine.

Worldwide governmental restrictions on cultivation, use of and research into cannabis meant that clinical process research work has not been sufficiently rigorous. Therefore, there exists a real need to work towards clearer understanding and development of the therapeutic activities of cannabis-based products and the development of optimum routes of administration for the purposes of efficacy and safety.

Although much of the research carried out to date has been directed towards the bioactivity of the phytocannabinoids, emphasis is increasingly being placed on the full spectrum of phytochemicals including terpenes and phenylpropanoids.

Cannabis based medicinal products (CBMPs) are defined as mixtures comprising one or more purified cannabinoids including THC, CBD and others. They are manufactured to Good Manufacturing Practice (GMP) standards and are compliant with all patient safety requirements. Only two CBMPs, based on purified isolates, currently have full market authorisation. In order to have market authorisation an unlicensed CBPM has to go through a New Drug Application (NDA) process. This is a long and tortuous process that starts with a formal application to the regulatory body and include full pharmacodynamic data and pharmacokinetic data as well as description of the manufacturing processes, analytical data relating to product purity and specification, and impurity profiles, batch-to-batch reproducibility and efficacy.

While cannabinoids and terpenes have been studied substantially for their individual therapeutic effects, there is a general consensus that CBPMs activate their pharmacological effects in the living body via synergistic/antagonistic interactions between the various phytochemicals that are present in the cannabis-based products and the endocannabinoid system (ECS). This phenomenon, also known as “entourage effect”, was coined to explain why botanical drugs containing the entire spectrum of compounds within a plant can be more effective than the plant's isolated components. A good example of the entourage effect is the stronger muscle-antispastic effect of cannabis extract compared to pure THC, which represents an important finding for the treatment of multiple sclerosis.

Traditional processes for extraction from biomasses may rely on the use of organic solvents such as ethanol or hexane and supercritical fluids such as carbon dioxide. Such processes have a common problem in that the solvents have poor selectivity and extracts require further downstream purification. GB 2393720A describes a general process for extraction of components from a biomass which aims to fractionally extract materials. The process involves producing different extracts by changing the physical state of a solvent formulation, changing the physical state of the biomass or changing the solvating power of the formulation, for example by mixing a main solvent with a co-solvent. However, it is found that the process described does not comply with the requirements that define good manufacturing practice (GMP) for production of botanical drug substances (BDSs) that would be required if the extracts were to be used as pharmaceutical materials. In addition, the extract produced are not optimised and may require downstream purification.

To overcome production problems, many producers market semi-purified extracts or an artificial “full spectrum” product that is produced by blending of a number of extracts. Such known production processes do not provide a primary extract which is a true full spectrum product.

In addition, known processes, such as those described in GB2393720B and others, have a serious drawback in that the processes described necessitate the use of a gas vapour compressor which makes it difficult to produce full-spectrum extracts which meet the stringent cleaning in process (CIP) requirement for pharmaceutical GMP processes that are compulsory in medical and pharmaceutical production applications.

There exists a need for a process which is environmentally benign, simple in terms of equipment engineering complexity and operation, highly efficient in the extraction of the desired molecules whilst being selective in that it does not co-extract unwanted molecules such as waxes and heavy molecular weight aromatic and flavoursome polyphenols. For a process to be GMP, it is also desirable that a process can be carried out without the use of a gas vapour compressor.

It is an object of the present invention to address the above described problems.

It is an object of the present invention to provide substantially full spectrum extracts using a pharmaceutically-acceptable GMP process.

According to a first aspect of the invention, there is provided apparatus for extracting components from a material of natural origin, the apparatus comprising:
(A) an extraction vessel for containing the material of natural origin, said extraction vessel including an inlet for introducing a solvent into the extraction vessel and an outlet for removing the solvent from the extraction vessel; and
(B) a temperature adjustment means for adjusting the temperature of the solvent upstream of said inlet, wherein said temperature adjustment means comprises:
(i) an electrically-powered heater; and/or
(ii) a heating means (XX) having an inlet and an outlet, wherein said heating means (XX) is arranged to increase the temperature of solvent passing from said inlet to said outlet by at least 10° C. in a time of less than 60 seconds.

Said heating means (XX) may be arranged to increase the temperature of solvent passing from said inlet to said outlet by at least 15° C. in a time of less than 60 seconds. Said heating means (XX) may be arranged to increase the temperature of solvent passing from said inlet to said outlet by at least 20° C. in a time of less than 60 seconds.

Said heating means (XX) is preferably arranged to increase the temperature of solvent passing from said inlet to said outlet by at least 10° C. in a time of less than 20 seconds. Said heating means (XX) may be arranged to increase the temperature of solvent passing from said inlet to said outlet by at least 15° C. in a time of less than 20 seconds. Said heating means (XX) may be arranged to increase the temperature of solvent passing from said inlet to said outlet by at least 20° C. in a time of less than 20 seconds.

Said heating means (XX) is preferably arranged to increase the temperature of solvent passing from said inlet to said outlet by at least 10° C. in a time of less than 10 seconds. Said heating means (XX) may be arranged to increase the temperature of solvent passing from said inlet to said outlet by at least 15° C. in a time of less than 10 seconds. Said heating means (XX) may be arranged to increase the temperature of solvent passing from said inlet to said outlet by at least 20° C. in a time of less than 10 seconds.

In a preferred embodiment, said heating means (XX) is arranged to increase the temperature of solvent passing from said inlet to said outlet by at least 15° C. in a time of less than 10 seconds. In an especially preferred embodiment, said heating means (XX) is arranged to increase the temperature of solvent passing from said inlet to said outlet by at least 20° C. in a time of less than 6 seconds.

Said heating mean may comprise an electrically-powered heater as described in B(i).

Said heating means (XX) may have a length (L1) between its inlet and outlet of at least 500 mm, preferably at least 750 mm, more preferably at least 900 mm. The length (L1) may be less than 2000 mm or less than 1500 mm.

The heating means (XX) may comprise a flow passageway between its inlet and outlet. (which preferably has a substantially constant cross-section along its length). The heating means (XX) suitably has a flow obstruction element to create turbulence. It may have the following parameters:
A) Cross sectional area (front area) between 175 mm²-1256 mm², preferably 315 mm²-706 mm²; and
B) Obstructed area between 115 mm²-804 mm², preferably 200 mm²-450 mm². The free flow area would be equal to A-B.

Said flow passageway may have a perimeter (P1) via which heat can be transferred from the heating means (XX).

Said heating means (XX) may comprise a heater resistance of 50-2000Ω/m, preferably 75-150Ω/m.

Said heating means (XX) may comprise a body which is suitably in contact with a wall which defines a flow passageway for solvent, said body having a surface temperature of at least 35° C., preferably at least 40° C., more preferably at least 45° C. Said surface temperature may be less than 60° C. or less than 50° C. In preferred embodiments, said surface temperature may be between 30° C.-70° C. or preferably between 40° C.-60° C.

Said heating means (XX) may be arranged to supply heating power of at least 0.4 KW, preferably at least 0.5 kW. The power supplied may be less than 1.0 KW and is preferably between 0.5 KW and 0.8 kW.

The apparatus is preferably operable for flow of solvent in said flow passageway of said heating means (XX) at a rate of at least 0.01 m³/hr, preferably at a rate of at least 0.015 m³/hr. Said rate may be less than 0.05 m³/hr.

Said heating means (XX) may comprise a coil of resistance wire.

Said apparatus may include control means for controlling operation of said temperature adjustment means, for example said heating means (XX), to adjust the amount of heat transferred, in use, to solvent, thereby to control the temperature of solvent, for example at said outlet of said heating means (XX).

Said apparatus may include a temperature sensor (T1) downstream of said temperature adjustment means, for example said heating means (XX), for monitoring the temperature of solvent, wherein, preferably, information relating to the temperature sensed is communicated to the control means.

Said extraction vessel is preferably downstream of said temperature adjustment means, for example said heating means (XX), and the apparatus is arranged for flow of solvent from said temperature adjustment means to said extraction vessel via a conduit (C1), which is suitably directly connected to the extraction vessel with no other vessel between the temperature adjustment means and the extraction vessel. There may, however, be one or more valves and/or sensors between the temperature adjustment means and the extraction vessel.

Said the extraction vessel may have a volume range in the range 1 litre and 50 litres, preferably in the range 21 to 251. The ratio diameter/length of the extraction vessel may be between 0.02/1 and 0.3/1, preferably between 0.4/1 and 0.2/1.

Said extraction vessel preferably includes heating means (XX) for heating the contents of the vessel. The heating means (XX) is preferably electrically powered and/or includes a resistance heating element arranged to transfer heat to an outside wall of the extraction vessel.

Said extraction vessel preferably includes a pressure sensor (P2) for sensing pressure in the extraction vessel, wherein, preferably, information relating to the pressure in the vessel is arranged to be communicated to the control means. Sensor (P2) is suitably arranged to measure pressure at the outlet of extraction vessel before a downstream valve (eg valve 36 described hereinafter) which is actuated by a signal from or initiated by sensor (P2). This allows the downstream valve to open proportionally so that the pressure within the extraction vessel is maintained at above the vapour pressure of the solvent.

Said extraction vessel preferably includes an inlet for solvent at its lower end and/or is arranged for flow of solvent upwards between its inlet and outlet.

Said apparatus preferably includes a solvent pum

for pumping solvent into and/or through the temperature adjustment means, for example said heating means (XX). Said pump is preferably arranged to pum

solvent at a rate of at least 10 litres/hour, preferably at least 15 litres/hour, and suitably, at a rate of less than litres/hour. In a preferred embodiment, said pump is preferably arranged to pump solvent at a rate of 10 times to 50 times the bed volume of material of natural origin in the extraction vessel.

Said apparatus preferably includes a solvent storage vessel, suitably upstream of said extraction vessel and suitably upstream of said temperature adjustment means, for example said heating means (XX). A conduit (C4) is suitably directly connected between the extraction vessel and said temperature adjustment means. Said temperature adjustment means is preferably between the extraction vessel and the solvent storage vessel.

Said apparatus preferably includes a temperature sensor (T2) downstream of said solvent storage vessel (and said sensor (T2) is preferably upstream of said temperature adjustment means, wherein said sensor (T2) is arranged to communicate information relating to the temperature sensed to the control means).

Said solvent storage vessel preferably includes a pressure sensor P3 for sensing pressure in the solvent storage vessel, wherein, preferably, information relating to the pressure in the vessel is arranged to be communicated to the control means.

Said solvent storage vessel preferably includes a temperature sensor (T3) for sensing the temperature of solvent in the solvent storage vessel, wherein said sensor (T3) is arranged to communicate information relating to the temperature sensed to the control means).

Said solvent storage vessel may have a volume of 1 to 2 times the total dead volume of the apparatus; and/or 1 to 2 times the sum of the volume of the extraction vessel and an evaporator vessel. Said solvent storage vessel preferably includes a cooling means, for example including a refrigeration unit which is suitably arranged to liquefy solvent in the solvent storage vessel.

Said apparatus preferably includes a said evaporator vessel, suitably upstream of said solvent storage vessel. Said evaporator vessel preferably includes a temperature sensor (T4) for sensing the temperature of solvent in the evaporator vessel, wherein said sensor (T4) is arranged to communicate information relating to the temperature sensed to the control means).

Said evaporator vessel may have a volume which is between 2+ and 20+, preferably 4+ and 10×, the volume of the extraction vessel.

Said evaporator vessel preferably includes a heating means (YY). Said heating mean (YY) may comprise an electrically-powered heater. Said heating means (YY) may be operated at 240V ac. Said heating means (YY) may have sufficient power to enable self-regulating to maintain the evaporator temperature in the evaporator vessel in the range 0° C. to 20° C. or preferably in the range 5° C. and 10° C.

Said heating means (YY) may be arranged to supply heating power of at least 0.1 kW. The power supplied may be less than 0.4 KW. Said heating means (YY) may comprise a resistance wire.

Said apparatus may include control means for controlling operation of said heating means (YY), to adjust the amount of heat transferred, in use, to said evaporator vessel, thereby to control the temperature of solvent in said vessel in use.

Said evaporator vessel preferably includes a pressure sensor (P5) for sensing pressure in the evaporator vessel, wherein, preferably, information relating to the pressure in the vessel is arranged to be communicated to the control means.

Said evaporator vessel preferably includes a temperature sensor (T5) for sensing the temperature of solvent in the evaporator vessel, wherein said sensor (T5) is arranged to communicate information relating to the temperature sensed to the control means.

Said apparatus preferably includes said extraction vessel and said evaporator vessel as described, wherein said extraction vessel is upstream of said evaporator vessel and, suitably, the apparatus is arranged for flow of solvent (and entrained extract of the material of natural origin) from the extraction vessel to the evaporator vessel, suitably via a conduit (C2) which is suitably directly connected to the evaporator vessel with no other vessel between the extraction vessel and the evaporator vessel. There may, however, be one or more valves and/or sensors between the extraction vessel and the evaporator vessel.

Said apparatus may include a temperature sensor (T4) between the extraction vessel and said evaporator vessel, wherein said sensor (T4) is arranged to communicate information relating to temperature sensed to the control means.

Said evaporator vessel may include an outlet (Z1) for removing components extracted from the material of natural origin from the apparatus and/or from the evaporator vessel, suitably after solvent has been evaporated from a mixture comprising solvent and such components.

Said evaporator vessel suitably includes an outlet (Z2) for passage of solvent from the evaporator vessel to the solvent storage vessel, suitably via a conduit (C3) which is suitably directly connected between the evaporator vessel and the solvent storage vessel with no other vessel between the evaporator vessel and the solvent storage vessel. There may, however, be one or more valves and/or sensors between the evaporator vessel and the solvent storage vessel.

Said apparatus preferably does not include a vapour compressor, for example in a solvent flow path from the evaporator vessel and back thereto, suitably via the solvent storage vessel, the temperature adjustment means and said extraction vessel.

In order to negate the necessity for a vapour compressor to remove solvent from the evaporator vessel and to liquefy it in the solvent storage vessel, the apparatus, for example the said control means, is suitably arranged to maintain temperature and pressure differentials between the evaporator vessel and the solvent storage vessel. Thus, the apparatus, for example said control means, may be arranged to control the supply of heat to the evaporator vessel so that a steady-state temperature of between 10°0 C. and 30°0 C. is maintained whilst cooling is applied to the solvent storage vessel, suitably to maintain a steady-state temperature of about −10° C. to −30° C. Thus, the apparatus is suitably arranged and/or the control means arranged to be operated so a temperature/pressure differential between the two vessels is arranged for the substantially continuous removal of solvent as vapour from the evaporator vessel to the solvent storage vessel.

The apparatus preferably includes:
a said temperature adjustment means, for example a heating means (XX), as described;
a said solvent storage vessel, as described;
a said extraction vessel, as described;
a said evaporator vessel, as described.

The apparatus preferably includes:
a said conduit (C1) between the temperature adjustment means and extraction vessel;
a said conduit (C4) between the solvent storage vessel and the temperature adjustment means;
a said conduit (C3) between the evaporator vessel and the solvent storage vessel;
a said conduit (C2) between the extraction vessel and the evaporator vessel, wherein preferably there is no vessel between the temperature adjustment means and the extraction vessel. Preferably, there is no vessel between the solvent storage vessel and the temperature adjustment means. Preferably, there is no vessel between the evaporator vessel and the solvent storage vessel. Preferably, there is no vessel between the extraction vessel and the evaporator vessel.

The apparatus preferably includes only one pump for pumping solvent around a fluid flow path defined from the extraction vessel and back, for example via the evaporator vessel, the solvent storage vessel and the temperature adjustment means.

In a preferred embodiment, said apparatus includes an extraction vessel which contains a material of natural origin.

Said material of natural origin may be used directly after harvesting without any chemical or physical treatment. Preferably, however, said material of natural origin is treated, suitably prior to introduction in the extraction vessel. It may be treated physically, for example dried and/or size reduced. The material of natural origin contained in the extraction vessel may have a water content of at least 5 wt % or at least 15 wt %; the water content is suitably less than 40 wt % or less than 25 wt %. The material of natural origin may have a particle of size less than 10 mm, for example less than 5 mm. At least 50 wt %, or at least 80 wt % of said material of natural origin may comprise particle with a maximum dimension of less than 10 mm.

Said material of natural origin may be treated to derivatise one or more components which are naturally present in the material of natural origin. For example, said material of natural origin may be a decarboxylated material of natural origin.

Preferably, in use, said extraction vessel is fully (and tightly) packed so that the packed bed is stationary. Non-botanical material that is inert, such as glass, may be used to ensure the space is completely packed. The density of packing may be between 0.3 kg/l-0.8 Kg/l, or 0.4 kg/l-0.7 Kg/l.

The apparatus may be used to treat a range of materials of natural origin and may be used to treat botanicals that have bioactive properties in general. In a preferred embodiment, said material of natural origin is a member of the cannabaceae plant family such as cannabis indica, ruderalis, sativa with cannabis sativa being preferred.

Advantageously, the apparatus can be used to produce pharmaceutically acceptable extracts which may be regarded as GMP. Thus, extracts may be classified as Botanical Drug Substances and/or to be authorised for use as pharmaceuticals.

In a preferred embodiment, the apparatus includes a solvent, for example contained in a said solvent storage vessel. In use, said solvent may also be present in said extraction vessel, said evaporator vessel and said solvent storage vessel.

Said solvent may comprise a C₁₋₄ fluorinated hydrocarbon. Said C₁₋₄ fluorinated hydrocarbon is preferably non-chlorinated. Preferably, it comprises one or more carbon atoms, one or more fluorine atoms together with one or more other atoms selected from hydrogen atoms and iodine atoms. More preferably, it comprises one or more carbon, fluorine and hydrogen atoms only. Preferably, said fluorinated hydrocarbon is a C₁₋₃, more preferably a C₂₋₃, fluorinated hydrocarbon. Especially preferred is a C₂ fluorinated hydrocarbon. Said fluorinated hydrocarbon may include 10 or fewer, suitably 8 or fewer, preferably 7 or fewer, more preferably 5 or fewer, especially 4 or fewer, fluorine atoms. Preferably, said fluorinated hydrocarbon includes at least 2, more preferably at least 3, fluorine atoms.

Said fluorinated hydrocarbon may be unsaturated, such as R1234yf. It is preferably aliphatic. It is preferably saturated.

Said fluorinated hydrocarbon may have a boiling point at atmospheric pressure of less than 20° C., preferably less than 10° C., more preferably less than 0° C., especially less than −10° C. The boiling point may be greater than −90° C., preferably greater than −70° C., more preferably greater than −50° C., especially greater than −40° C.

Said solvent may comprise a solvent selected from: iodotrifluoromethane, CF₃H (HFC-23, trifluoromethane), CH₃F (HFC-41, fluoromethane), CH₂F₂ (HFC-32, difluoromethane), CF₃CF₂H (HFC-125, pentafluoroethane), CF₃CH3 (HFC-143 A, 1,1,1-trifluoroethane), HCF₂CH₃ (HFC-152 A, 1,1-difluoroethane), CF₃CHFCF₃ (HFC-227 EA, 1,1,1,2,3,3,3-heptafluoropropane), CF₃CF₂CF2H (HFC-227 CA, 1,1,1,2,2,3,3-heptafluoropropane), CF₃CH₂CF₃ (HFC-236 FA, 1,1,1,3,3,3-hexafluoropropane), CF₃CF₂CH₃ (HFC-245 CB, 1,1,1,2,2-pentafluoropropane), CF3CF2CH2F (HFC-236 CB, 1,1,1,2,2,3 -hexafluoropropane), HCF₂CF₂CF₂H (HFC-236 CA, 1,1,2,2,3,3-hexafluoropropane), CF₃CHFCF₂H (HFC-₂₃₆ EA, 1,1,1,2,3,3-hexafluoropropane), and CH₂FCF₃ (HFC-134A, 1,1,1,2-tetrafluoroethane).

Preferably, said solvent comprises a solvent selected from: iodotrifluoromethane, 1,1,1,2,3,3,3-heptafluoropropane (HFC-227 EA), 1,1,1,2,2,3,3-heptafluoropropane (HFC-227CA) and 1,1,1,2-tetrafluoroethane (HFC-134a).

More preferably, said solvent comprises a solvent selected from: 1,1,1,2,3,3,3-heptafluoropropane (R-227EA) and 1,1,1,2-tetrafluoroethane, with 1,1,1,2-tetrafluoroethane being especially preferred.

Said C₁₋₄ fluorinated hydrocarbon preferably has a purity of at least 98% w/w.

Said solvent preferably comprises at least 80 wt %, preferably at least 95 wt %, more preferably at least 99 wt % of said C₁₋₄ fluorinated hydrocarbon and preferably comprises at least 99 wt % of a single type of C₁₋₄ fluorinated hydrocarbon. Said solvent preferably consists essentially of a C₁₋₄ fluorinated hydrocarbon which is preferably 1, 1,2-tetrafluoroethane.

Said apparatus preferably includes a control means which receives information from one or more (preferably each) of said temperature sensors T1 to T4 and/or one or more of said pressure sensors P1, P2, P3, P5. The apparatus may be arranged to control heat or cooling of the solvent storage vessel and/or the evaporator vessel, depending upon the temperature and pressure sensed by said one or more sensors.

The apparatus is preferably a closed-loo

system and, preferably, it includes only one mechanical element within the closed loop, namely said pump (which is suitably upstream of said temperature adjustment means). The arrangement means that the risk of contamination of extracts produced using the apparatus is minimised and efficient cleaning-in-process (CIP) can be readily accomplished.

According to a second aspect of the invention, there is provided a process for extracting components from a material of natural origin, the process comprising:
(a) selection of apparatus according to the first aspect;
(b) with a material of natural origin, as described herein, in the extraction vessel, operating the apparatus to introduce solvent into the extraction vessel to contact said material of natural origin;
(c) removing solvent from the extraction vessel wherein said solvent includes any components extracted from the material of natural origin and directing said solvent to an evaporator vessel;
(d) evaporating solvent in the evaporator vessel; and
(e) collecting components extracted from the material of natural origin.

The apparatus used in the process suitably includes an evaporator vessel and a solvent storage vessel and the method preferably comprises maintaining temperature and pressure differentials between the evaporator vessel and the solvent storage vessel so that solvent is substantially continuously removed as vapour from the evaporator vessel, with said vapour passing to the solvent storage vessel where it is preferably condensed to a liquid. This process suitably negate the necessity for a vapour compressor to remove solvent from the evaporator vessel and to liquefy it in the solvent storage vessel. The method preferably does not include use of a vapour compressor to reliquefy solvent.

In the process, the temperature T2 is suitably maintained substantially constant during operation of the process.

In a preferred embodiment, control means of the apparatus is operated in the process to control the supply of heat to the evaporator vessel so that a steady-state temperature of between 10° C. and 30° C. is maintained whilst cooling is applied to the solvent storage vessel, suitably to maintain a steady-state temperature of about −10° C. to −30° C. Thus, the apparatus is suitably arranged and/or the control means arranged to be operated so a temperature/pressure differential between the two vessels is arranged for the substantially continuous removal of solvent as vapour from the evaporator vessel to the solvent storage vessel.

Preferably the process is a GMP process. Said process is preferably for producing substantially full spectrum extracts (eg BDS extracts) using a pharmaceutically-acceptable GMP process.

Preferably, in the process, said temperature adjustment means, for example, said heating means (XX) is operated to increase the temperature of solvent passing from its inlet to its outlet by at least 10° C. in a time of less than 60 seconds.

In a preferred embodiment, control means of the apparatus is operated to control the temperature of solvent which contacts the material of natural origin in the extraction vessel, with the preferred temperature being in the range 10° C.-30° C. In light of this, the temperature adjustment means is arranged to rapidly increase the temperature of solvent between the solvent storage vessel and the extraction vessel.

Any feature or any aspect of any invention or embodiment described herein may be combined with any feature of any aspect of any other invention or embodiment described herein mutatis mutandis.

Specific examples of the present invention will now be described, by way of example, with reference to the following figures, in which:

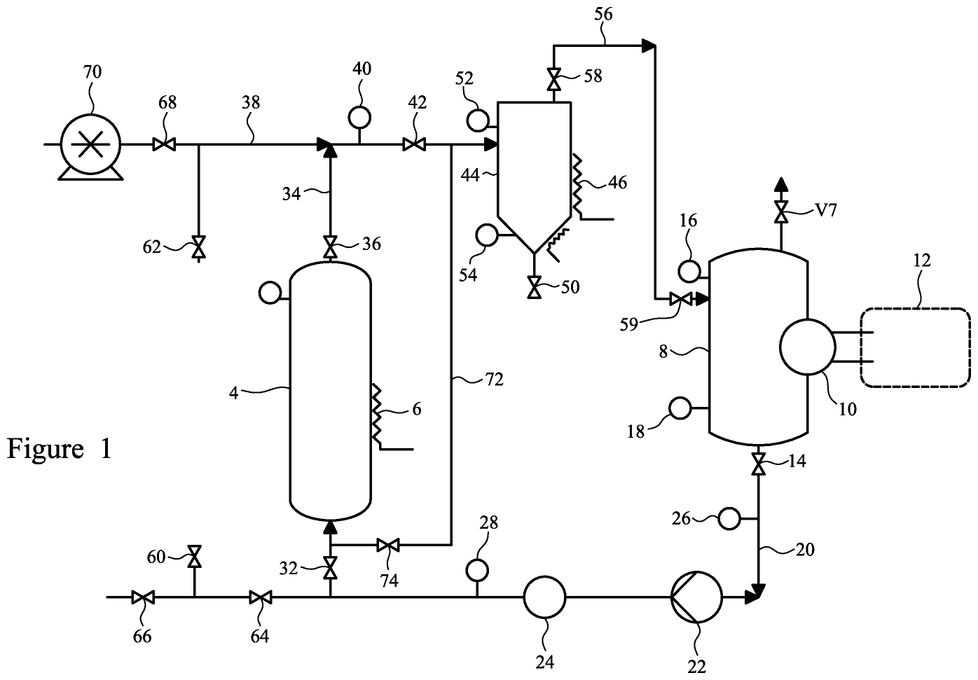

FIG. 1 is a schematic representation of apparatus for extraction from a biomass; and

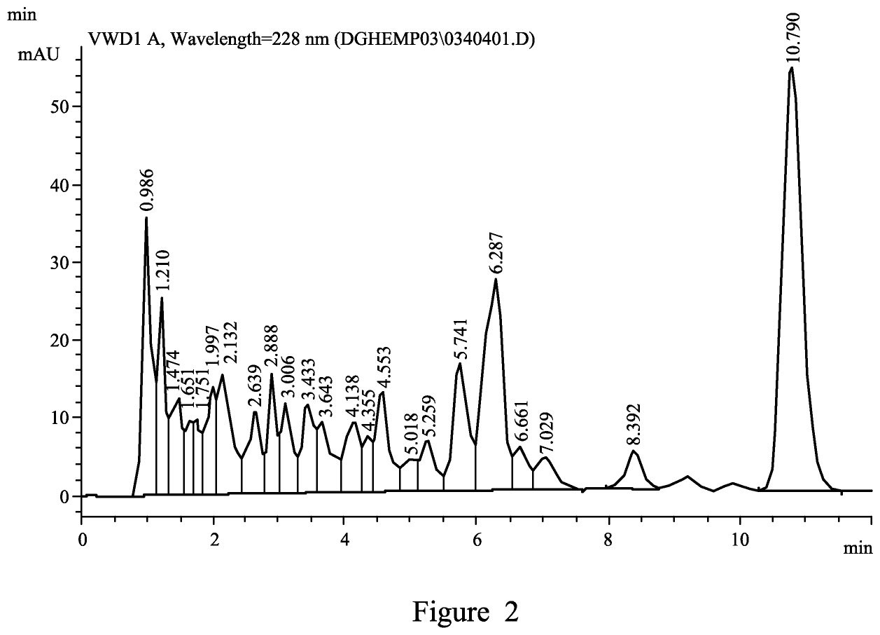

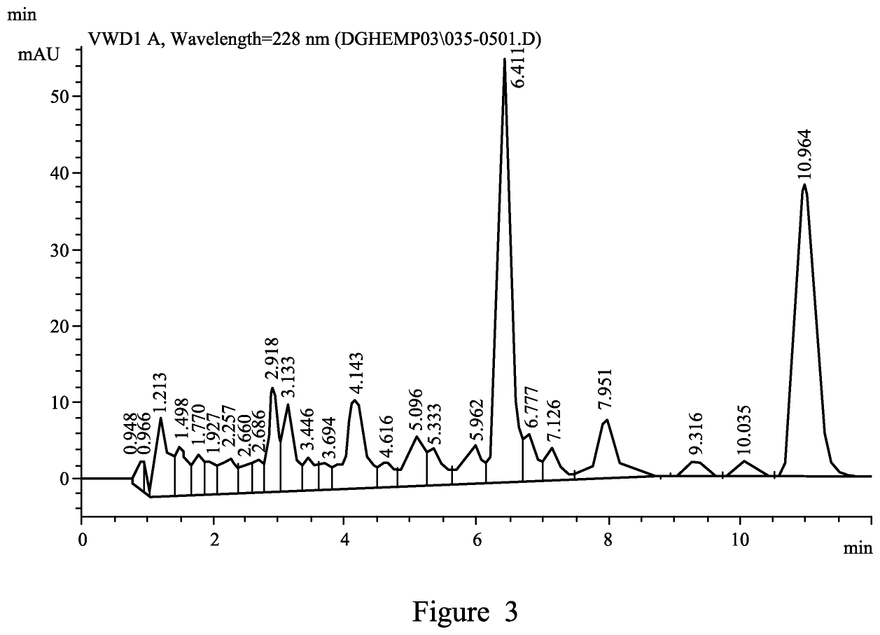

FIGS. 2 and 3 are HPLC chromatograms of extracts produced using ethanol and HFC134a respectively.

The following is referred to hereafter:

HFC134a—refers to 1, 1, 1, 2-tetrafluoroethane.

Apparatus 2 for extraction of a biomass comprises an extraction vessel 4 for containing a biomass to be extracted. Electrical heating tape or an electrical heating blanket (shown schematically and referenced 6) is provided around and in contact with the vessel 4 for maintaining the temperature of the wall of vessel 4 and, consequently, the contents therein, within the desired limits.

Upstream of vessel 4 is a solvent recycling vessel 8 which is fitted with a cooling coil (shown schematically by reference numeral 10) which communicates with an external refrigeration unit 12. Operation of unit 12 is arranged to cool (and thereby liquefy) solvent in the vessel 8. A pressure sensor 16 and a temperature sensor 18 are provided for monitoring temperature and pressure within vessel 8.

A pipe 20 communicates with an outlet of vessel 8 and is arranged to transport solvent to a solvent pump 22. Valve 14 is provided adjacent to the outlet of vessel 8 for controlled passage of solvent from vessel 8 into pipe 20.

The solvent pum

22 is arranged to pum

liquid between vessel 8 and vessel 4 via an electrically powered heat exchanger 24. A temperature sensor 26 is arranged to monitor temperature of solvent in pipe 20.

The heat exchanger 24 is arranged to increase the temperature of solvent flowing through it very rapidly. For example, it is suitably arranged to increase temperature of solvent between its inlet and outlet from as low as −30° C. to as high as 30° C. in less than 1 minute. This allows the temperature of solvent introduced into extraction vessel 4 to be very rapidly changed in a step-wise manner rather than temperature of solvent being changed linearly over an extended period of time. This is found to significantly affect speed, efficiency and controllability of extraction processes using the apparatus and facilitates production of higher quality extracts.

The heat exchanger may have a working length between its inlet and outlet of between 800 mm and 1400 mm. The pipe with which the heat exchanger is associated may have an internal diameter of between 6 mm and 20 mm.

The heat exchanger is electrically operated, since such operation has been found to be able to produce a sufficiently rapid temperature increase of solvent passing through. Said heat exchanger may have a heater resistance of about 100Ω per metre; it may be operated at 240V ac.

Downstream of the heat exchanger 24 is a temperature sensor 28 for monitoring temperature of solvent in pipe 30, after passage through the heat exchanger.

Pipe 30 is arranged to transport solvent into vessel 4 via a valve 32.

Vessel 4 may have a volume of 3L-5L for containing biomass to be extracted.

A pipe 34, in which valve 36 is arranged, is provided downstream of vessel 4. Pipe 34 communicates with a pipe 38 which is arranged to transport solvent via temperature sensor 40 and valve 42 to an evaporator vessel 44. This vessel 44 is wrapped with electrical heating tape or an electric heating blanket (shown schematically and referenced 46). Vessel 44 includes a pressure sensor 52 and a temperature sensor 54. It also includes an outlet and associated valve 50 via which extract can be drawn off from the apparatus 2.

Valve 36 incorporates a pressure sensor so that a pressure differential between vessel 4 and vessel 44 is maintained. This mechanism to ensure that the pressure within vessel 4 is always kept above the vapour pressure so that the solvent medium within vessel 44 is maintained in a liquid state.

A pipe 56 is arranged to transport solvent back to vessel 8 via valves 58, 59.

Also illustrated in FIG. 1 are valves 60, 62, 64, 66 and 68 which may be associated with and/or arranged to control passage of solvent to/from a second extraction vessel (not shown) which may be as described for vessel 4 and may be positioned between valves 60, 62.

The apparatus also includes a vacuum pum

70 for facilitating vacuum aided evaporation of solvent in evaporator vessel 44.

In addition, the apparatus includes a shunt pipe 72 and associated valve 74 for returning solvent from pipe 38 to extraction vessel 4, if required at the end of the extraction run.

In general terms, the apparatus 2 may be operated as follows.

A biomass to be extracted is packed into extraction vessel 4. Then, vacuum pump 70 is operated to remove air from the apparatus. With HFC134a in vessel 8, refrigeration unit 12 is used to maintain the solvent at a suitable temperature and pressure in the liquid state, with the state of the solvent being monitored by pressure and temperature sensors 16, 18.

The solvent is pumped from vessel 8, in pipe 20, by pum

22 and the temperature of the solvent is monitored by temperature sensor 26. The solvent then passes into and through the heat exchanger 24.

The heat exchanger 24 is operated to rapidly increase the temperature of the solvent as may be required during extraction of biomass. Typically, the solvent enters the heat exchanger at a temperature in the range −30° C. to −10° C. In one embodiment, the heat exchanger may be operated simply to maintain this low temperature in which case the temperature of solvent measured by temperature sensor 28 may be the same as that measured by sensor 26. Such a low temperature may be used to extract the most soluble components in the biomass. Alternatively, or additionally, the heat exchanger may be operated to very rapidly increase the temperature of solvent passing through it. For example, the heat exchanger may be operated to produce a temperature rise of 10 to 50° C. in less than 1 minute. In one embodiment, the heat exchanger may be operable to increase the solvent temperature between 10 and 50° C. per metre of heater exchanger in less than 1 minute or even less than 20 seconds.

In one embodiment the extraction may be carried out under isocratic temperature conditions.

Vessel 4 temperature is either a step-wise gradient as described in the table, or a steady state temperature that may be pre-set somewhere between 10° C. and 3° C. The temperature in vessel 44 is maintained in the steady state at predetermined anywhere between 20 and 30° C. Similarly, temperature in vessel 8 is suitably at a steady state as described.

From the heat exchanger 24, the solvent is pumped via pipe 30 and valve 32 into extraction vessel 4. It will be appreciated that the temperature of the solvent entering vessel 4 affects what is extracted from the biomass. At lower solvent temperatures, only the most soluble constituents will be extracted from the biomass; as the solvent temperature increases, a more complex mixture of components, which will be more concentrated in less soluble constituents, will be extracted. Advantageously, it is found that the ability to rapidly change the temperature of the solvent can be manipulated to produce higher quality extracts in contrast to situations wherein solvent temperature is changed more slowly over a relatively prolonged period which is the case with the apparatus of GB2393720B.

Solvent may be passed through the biomass at a rate of 30-70 L/hour or more ideally 40-60 L/hour. The solvent and entrained constituents extracted from the biomass exit vessel 4 at its upper end and pass via valves 36, 42 and pipes 34, 38 to the evaporator vessel 44. The temperature (and pressure) of the liquid in vessel 4 are monitored by temperature and pressure sensors 52, 54 and temperature adjusted, as necessary, by operation of electrical heating blanket/tape 46. Consequently, the HFC134a solvent is evaporated and transferred via valve 58, pipe 56 and valve 59 back to the recycling vessel 8. Valve 36 is electronically controlled by a pressure signal so that it opens and closes whilst keeping the pressure within vessel 8 at a predetermined level above that of the vapour pressure of the solvent. This ensures the liquid status of the solvent within the vessel 8 is maintained.

The vessel 8 is cooled by refrigeration unit 12 so as to maintain sufficient vapour pressure differential between vessels 44 and 8 so that simultaneous evaporation of the HFC134a solvent in vessel 44 and re-condensing in vessel 8 occurs continuously. This is made possible by the ability to accurately and rapidly monitor temperature and pressure in vessels 44 and 8 and the ability to rapidly adjust the temperature of liquid in vessel via electrical heating blanket/tape 46.

After evaporation of HFC134a solvent, extract remains in the evaporation vessel 44 from which it can be drawn off via valve 50. The selectivity of this process, inherent in the choice of solvent as well as the optimum extraction conditions, means that the required molecules, including the cannabinoids and terpenes, are efficiently extracted whilst most of the unwanted molecules, which mostly are plant waxes and heavy molecular weight polyphenols, are not extracted. The extract obtained via this process does not require further downstream purification steps such as winterisation and/or chromatography. Therefore, the extract obtained may be described as a primary and full spectrum with respect to the desired molecules which are primarily cannabinoids and terpenes.

Advantageously, the apparatus does not need a compressor to re-liquefy the solvent and this fact enables the apparatus to be operated according to GMP thereby allowing the extracts to be classified as Botanical Drug Substances and/or to be authorised for use as pharmaceuticals.

The biomass used in the apparatus is suitably cannabis sativa, although the apparatus may be used to produce full spectrum extracts from other members of the cannabaceae plant family such as cannabis indica and ruderalis.

The following examples further illustrate the invention.

### Example 1—Preparation of Cannabis sativa for Extraction

Cannabis sativa biomass was dried to a water content of 20 wt %, milled to an average particle size of 1-3 mm and decarboxylated using standard thermal methods.

### Example 2 (Comparative)—Ethanol Extraction

Decarboxylated biomass of Example 1 (ca. 5 g) was subjected to a standard soxhlet extraction with ethanol for 1.0 hour.

### Example 3—Extraction using the Apparatus of FIG. 1

Decarboxylated biomass (ca. 90 g) was packed into a stainless-steel extraction vessel 4 in the form of a column having dimensions of 32 mm/500 mm, compacted using mechanical means into a tightly packed bed and assembled with the other parts of the apparatus as shown in FIG. 1. With valves 64, 68, 62 and 7 closed, all the constituent elements of the apparatus were evacuated using the vacuum pum

70. With valves 14 and 59 closed, the solvent vessel 8 was charged with 4 Kg HFC134a via valve 7 and cooled down and maintained within the temperature range of −30° C. and −70° C. throughout the subsequent extraction operation. With valves 14, 32, 36 and 42 opened, the solvent pump 22 was started so that cold HFC134a passed via the heat exchanger 24 into the extraction vessel 4 in an up-flow mode so that mass transfer of some constituent compounds from the biomass into the solvent was caused to occur. The now “rich” HFC134a solution containing the extracted compounds was continuously directed via valves 36 and 42 into the evaporator 44, where it was heated using the electrically operated jacket causing evaporation of the solvent. The resulting pressure differential between the evaporator vessel 44 and the solvent vessel 8 caused the solvent vapour to flow into the solvent 8 where it was continuously reliquefied prior to recycling through the biomass whilst the extracted compounds were collected in the evaporator vessel. A steady state was maintained as follows for 1.5 hours, the operating parameters, with reference to FIG. 1, being as follows:
temperature sensor 28=15° C.-25° C.
temperature sensor 54=15° C.-25° C.; pressure sensor 5232 4.5 bar-5.5 bar
temperature sensor 18=−30° C.; pressure sensor 16=1.0 bar-2.5 bar.

At the end of the extraction, the solvent pum

22 was switched off, valve 14 closed and valve 74 opened. Temperature and pressure steady state were maintained until all the HFC134a was transferred via the evaporator vessel into the solvent storage vessel. The extracted product collected in the evaporator vessel was harvested, weighed and analysed by gradient HPLC.

### Examples 4 to 7—Treatment of THC Rich Biomass

### Example 4 (Comparative)

THC rich biomass was decarboxylated by placing the biomass in an oven at 90° C. for 2 hours and then treated as described in Example 2.

### Example 5 (Comparative)

THC rich biomass was vacuum packed, decarboxylated by autoclave at 129° C. for 2 hours and then treated as described in Example 2.

### Example 6

THC rich biomass was decarboxylated by placing the biomass in an oven at 90°0 C. for 2 hours and then treated as described in Example 3.

### Example 7

THC rich biomass was vacuum packed, decarboxylated by autoclave at 129° C. for 2 hours and then treated as described in Example 3.

Results for Examples 4 to 7 are provided in the table below

| | Biomass Weight | Extract Weight |
|---|---|---|
| Example No. | (g) | (g) |
| Example 4 | 5.0 | 1.61 |
| Example 5 | 5.0 | 1.47 |
| Example 6 | 91.3 | 10.92 |
| Example 7 | 86.28 | 9.30 |

### Examples 8 to 11—Treatment of CBD Rich Biomass

### Example 8 (Comparative)

CBD rich biomass treated as described in Example 4.

### Example 9 (Comparative)

CBD rich biomass treated as described in Example 5.

### Example 10

CBD rich biomass treated as described in Example 6.

### Example 11

CBD rich biomass treated as described in Example 7.

Results for Examples 8 to 11 are provided in the table below

| | Biomass Weight | Extract Weight |
|---|---|---|
| Example No. | (g) | (g) |
| Example 8 | 5.7 | 1.04 |
| Example 9 | 5.4 | 1.59 |
| Example 10 | 90.3 | 2.10 |
| Example 11 | 86.28 | 2.65 |

In Examples 4 to 11, a marked difference was noted between extracts produced using ethanol and HFC134a in terms of both physical texture and colour. Extracts produced by treating both THC and CBD rich biomasses using ethanol had a thick, gummy consistency and were dark greenish in colour. Extracts produced by treating both THC and CBD rich biomasses using HFC134a and the apparatus described had an oily consistency and were light yellow/light brown in colour. HPLC chromatograms are provided in FIGS. 2 and 3 which show marked differences which indicate a higher impurities profile for the ethanol extracts in comparison to the HFC134a.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. Apparatus for extracting components from a material of natural origin, the apparatus comprising:
a) an extraction vessel for containing the material of natural origin, said extraction vessel including an inlet for introducing a solvent into the extraction vessel and an outlet for removing the solvent from the extraction vessel; and
b) a temperature adjustment means for adjusting the temperature of the solvent upstream of said inlet, wherein said temperature adjustment means comprises:
i. an electrically-powered heater; and/or
ii. a heating means (XX) having an inlet and an outlet, wherein said heating means (XX) is arranged to increase the temperature of solvent passing from said inlet to said outlet by at least 10° C. in a time of less than 60 seconds.

2. Apparatus according to claim 1, wherein said heating means (XX) is arranged to increase the temperature of solvent passing from said inlet to said outlet by at least 15° C. in a time of less than 60 seconds; and/or said heating means (XX) is arranged to increase the temperature of solvent passing from said inlet to said outlet by at least 10° C. in a time of less than 20 seconds; and/or said heating means (XX) is arranged to increase the temperature of solvent passing from said inlet to said outlet by at least 10°0 C. in a time of less than 10 seconds; and/or said heating means (XX) is arranged to increase the temperature of solvent passing from said inlet to said outlet by at least 20° C. in a time of less than 6 seconds. cm 3. Apparatus according to claim 1 wherein said heating mean comprises an electrically-powered heater as described in b(i) which optionally includes a heater resistance of 50-200Ω/m; and/or said heating means (XX) has a length (L1) between its inlet and outlet of at least 500 mm and of less than 1500 mm. cm 4. Apparatus according claim 3, wherein said heating means (XX) comprises a body which is in contact with a wall which defines a flow passageway for solvent, said body having a surface temperature of at least 35° C.; and/or said heating means (XX) is arranged to supply heating power of at least 0.4 KW; and/or the apparatus is operable for flow of solvent in said flow passageway of said heating means (XX) at a rate of at least 0.01 m³/hour and suitably less than 0.05 m³/hour. cm 5. Apparatus according to claim 4, wherein said apparatus includes control means for controlling operation of said heating means (XX), to adjust the amount of heat transferred, in use, to solvent, thereby to control the temperature of solvent at said outlet of said heating means (XX); and wherein said apparatus includes a temperature sensor (T1) downstream of said heating means (XX), for monitoring the temperature of solvent, wherein information relating to the temperature sensed is communicated to a or said control means.

6. (canceled)

7. Apparatus according to claim 1, wherein said extraction vessel is downstream of said temperature adjustment means, for example said heating means (XX), and the apparatus is arranged for flow of solvent from said temperature adjustment means to said extraction vessel via a conduit (C1), which is directly connected to the extraction vessel with no other vessel between the temperature adjustment means and the extraction vessel, wherein, optionally said extraction vessel has a volume in the range 1 litre to 50 litres.

8. (canceled)

9. Apparatus according to claim 1, wherein said extraction vessel includes heating means (XX) for heating the contents of the vessel, wherein said heating means (XX) is electrically powered and/or includes a resistance heating element arranged to transfer heat to an outside wall of the extraction vessel; and said extraction vessel includes a pressure sensor (P2) for sensing pressure in the extraction vessel, wherein information relating to the pressure in the vessel is arranged to be communicated to a control means. cm 10. Apparatus according to claim 9, wherein said extraction vessel includes an inlet for solvent at its lower end and/or is arranged for flow of solvent upwards between its inlet and outlet. cm 11. Apparatus according to claim 1, wherein said apparatus includes a solvent pump for pumping solvent into and/or through the temperature adjustment means, for example said heating means (XX), wherein said pump is arranged to pump solvent at a rate of 10 times to 50 times a bed volume of material of natural origin in the extraction vessel. cm 12. Apparatus according to claim 1, wherein said apparatus includes a solvent storage vessel, upstream of said extraction vessel and upstream of said temperature adjustment means, wherein a conduit (C4) is directly connected between the extraction vessel and said temperature adjustment means, wherein said solvent storage vessel includes a cooling means which is arranged to liquefy solvent in the solvent storage vessel, wherein, optionally, said apparatus includes a temperature sensor (T2) downstream of said solvent storage vessel, wherein said sensor (T2) is arranged to communicate information relating to the temperature sensed to a control means; and said solvent storage vessel includes a pressure sensor P3 for sensing pressure in the solvent storage vessel, wherein information relating to the pressure in the vessel is arranged to be communicated to said control means; and said solvent storage vessel includes a temperature sensor (T3) for sensing the temperature of solvent in the solvent storage vessel, wherein said sensor (T3) is arranged to communicate information relating to the temperature sensed to the control means.

13. (canceled)

14. (canceled)

15. Apparatus according to claim 1, wherein said apparatus includes an evaporator vessel, wherein said evaporator vessel includes a temperature sensor (T4) for sensing the temperature of solvent in the evaporator vessel, wherein said sensor (T4) is arranged to communicate information relating to the temperature sensed to a control means; and said evaporator vessel includes a heating means (YY) which is an electrically-powered heater which is operable to maintain an evaporator temperature in the evaporator vessel in the range −0° C. to 20 C., wherein, optionally, said apparatus includes control means for controlling operation of said heating means (YY), to adjust the amount of heat transferred, in use, to said evaporator vessel, thereby to control the temperature of solvent in said vessel in use.

16. (canceled)

17. Apparatus according to claim 1, wherein said apparatus does not include a vapour compressor. cm 18. Apparatus according to claim 1, wherein said apparatus includes:
said temperature adjustment means, for example a heating means (XX);
a or said solvent storage vessel;
said extraction vessel;
a or said evaporator vessel;
a or said conduit (C1) between the temperature adjustment means and extraction vessel;
a or said conduit (C4) between the solvent storage vessel and the temperature adjustment means;
a or said conduit (C3) between the evaporator vessel and the solvent storage vessel;
a or said conduit (C2) between the extraction vessel and the evaporator vessel, wherein there is no vessel between the temperature adjustment means and the extraction vessel; and/or there is no vessel between the solvent storage vessel and the temperature adjustment means; and/or there is no vessel between the evaporator vessel and the solvent storage vessel; and/or there is no vessel between the extraction vessel and the evaporator vessel.

19. Apparatus according to claim 1, wherein the apparatus includes only one pump for pumping solvent around a fluid flow path defined from the extraction vessel and back, for example via a or said evaporator vessel, a or said solvent storage vessel and a or said temperature adjustment means.

20. Apparatus according to claim 1, wherein said apparatus includes an extraction vessel which contains a material of natural origin which is a member of the cannabaceae plant family and wherein the apparatus includes a solvent which comprises a C₁₋₄ fluorinated hydrocarbon.

21. (canceled)

22. Apparatus according to claim 1, wherein said apparatus includes a control means which receives information from one or more temperature sensors and/or one or more of pressure sensors and the apparatus is arranged to control heat or cooling of a solvent storage vessel and/or an evaporator vessel, depending upon the temperature and pressure sensed by said one or more sensors.

23. Apparatus according to claim 1, wherein the apparatus is a closed-loop system which includes only one mechanical element within the closed loop, wherein said mechanical element is a pump.

24. A process for extracting components from a material of natural origin, the process comprising:
a) selection of apparatus according to claim 1;
b) with material of natural origin in the extraction vessel, operating the apparatus to introduce solvent into the extraction vessel to contract said material of natural origin;
c) removing solvent from the extraction vessel wherein said solvent includes any components extracted rom the material of natural origin and directing said solvent to an evaporator vessel;
d) evaporating solvent in the evaporator vessel; and
e) collecting components extracted from the material of natural origin.

25. A process according to claim 24, wherein:
the apparatus used in the process includes an evaporator vessel and a solvent storage vessel and the method comprises maintaining temperature and pressure differentials between the evaporator vessel and the solvent storage vessel so that solvent is substantially continuously removed as vapour from the evaporator vessel, with said vapour passing to the solvent storage vessel where it is condensed to a liquid; and wherein control means of the apparatus is operated in the process to control the supply of heat to the evaporator vessel so that a steady-state temperature of between 10° C. and 30° C. is maintained whilst cooling is applied to the solvent storage vessel; and the process is a GMP process.

26. (canceled)

27. (canceled)

28. Apparatus for extracting components from a material of natural origin, the apparatus comprising:
(A) an extraction vessel for containing the material of natural origin, said extraction vessel including an inlet for introducing a solvent into the extraction vessel and an outlet for removing the solvent from the extraction vessel; and
(B) a temperature adjustment means for adjusting the temperature of the solvent upstream of said inlet, wherein said temperature adjustment means comprises:
(i) an electrically-powered heater which is arranged to increase the temperature of solvent passing from said inlet to said outlet by at least 10° C. in a time of less than 60 seconds;
wherein said heater has a heater resistance of 50-200Ω/m;
wherein said apparatus includes an extraction vessel which contains a material of natural origin which is a member of the cannabaceae plant family and wherein the apparatus includes a solvent which comprises a C₁₋₄ fluorinated hydrocarbon.
